(19) United States
(12) Patent Application Publication
(10) Pub. No.: US 2006/0123514 A1
(43) Pub. Date: Jun. 8, 2006

(54) Self-fertile apple resulting from S-RNAase gene silencing

(76) Inventors: Greet Janssens, Linden (BE); Willem Broekaert, Dilbeek (BE); Iise Van Nerum, Tielt-Winge (BE); Wim Broothaerts, Hoegaarden (BE); Rozemarijn Dreesen, Sint-Amandsberg (BE); Mark Davey, Gent (BE); Johan Keulemans, Korbeek-Lo (BE)

Correspondence Address: DILWORTH & BARRESE, LLP, 333 EARLE OVINGTON BLVD., UNIONDALE, NY, 11553, US

(21) Appl. No.: 11/250,004

(22) Filed: Oct. 13, 2005

Related U.S. Application Data
(60) Provisional application No. 60/618,111, filed on Oct. 13, 2004.

(51) Int. Cl. A01H 1/00; A01H 5/00; C12N 15/82

(52) U.S. Cl. 800/285; 800/315

(57) ABSTRACT

A transgenic plant or tree of Malus sp. comprising tissue derived from cells transformed with a nucleic acid molecule encoding a gametophytic S-RNAse from Malus sp. is provided. The nucleic acid molecule encoding a gametophytic S-RNAase is in a sense or antisense orientation relative to a regulatory region operably linked thereto. The regulatory region controls expression of the nucleic acid molecule, and the transgenic plant or tree exhibits self-compatibility compared to a corresponding non-transformed plant or tree.

### CROSS REFERENCE TO RELATED APPLICATIONS

This application claims priority to U.S. Provisional Application Ser. No. 60/618,111, filed Oct. 13, 2004, the contents of which are incorporated by reference herein.

### FIELD OF THE INVENTION

The present invention relates to the production of transgenic apple plants using S-gene alleles that control self-compatibility and the progeny, and harvestable parts thereof.

### BACKGROUND OF THE INVENTION

Fruit production in many tree fruit crops is dependent on cross-pollination between cultivars. This is due to the existence of a self-incompatibility (SI) mechanism, which is a widespread intraspecific system to prevent self-fertilization that is controlled by a single S-locus (deNettancourt 2001). Cross-pollination between compatible cultivars depends on insects as pollen vectors during flowering, and their activity is impaired by inclement weather. Suboptimal pollination efficiencies are one of the factors contributing to low annual fruit crops that may occur during certain years in commercial orchards (Goldway et al. 1999). There is a strong interest in the self-fertile character in many fruit and nut tree crops because self-pollination could ensure more consistently high production yields compared to cross-pollination. This has been apparent in sweet cherry and almond, where cultivars with dysfunctional SI genes have been obtained through mutagenesis and interspecific crosses, respectively (Godini et al. 1998). Although the severity of the SI reaction varies between cultivars and pollination conditions, true self-fertile apple cultivars are commercially non-existent.

Therefore, there is a commercial need for true self-fertile apple cultivars and nut tree crops because self-pollination would ensure more consistently high production yields compared to cross-pollination. The invention described herein uses a transgenic approach for inactivating the SI mechanism in apple to obtain self-fertile trees.

### SUMMARY OF THE INVENTION

One aspect of the invention is directed to a transgenic plant or tree of Malus sp. comprising tissue derived from cells transformed with a nucleic acid molecule encoding a gametophytic S-RNAse from Malus s. The nucleic acid molecule encoding a gametophytic S-RNAase is in a sense or antisense orientation relative to a regulatory region to which it is operably linked. The regulatory region controls expression of the nucleic acid molecule. The resulting transgenic plant or tree exhibits self-compatibility compared to a corresponding non-transformed plant or tree.

Another aspect of the invention is directed to a transgenic plant or tree wherein the gametophytic S-RNAase is encoded by an S-locus gene. The S-locus gene may be an S₃ or S₅ allele wherein only the 5′ part of the S-locus gene is in an antisense orientation relative to the regulatory region. In the alternative, only the 3′ portion of the S-locus gene is in an antisense orientation relative to the regulatory region. In still another aspect of the invention the S-locus gene has the nucleotide sequence as set forth in SEQ ID NO.: 1 (FIG. 5).

The invention is also directed to a transgenic plant or tree of Malus domestica comprising tissue derived from cells transformed with a nucleic acid molecule encoding a gametophytic S-RNAse from Malus s. The nucleic acid molecule encoding a gametophytic S-RNAase is in a sense or antisense orientation relative to a regulatory region to which it is operably linked. The regulatory region controls expression of the nucleic acid molecule. The resulting transgenic plant or tree exhibits self-compatibility compared to a corresponding non-transformed plant or tree.

As in the transgenic plant or tree of Malus sp. described above, the transgenic plant or tree of Malus domestica may comprise a gametophytic S-RNAase encoded by an S-locus gene. The S-locus gene may be an S₃ or S₅ allele wherein only the 5′ part of the S-locus gene is in an antisense orientation relative to the regulatory region. In the alternative, only the 3′ portion of the S-locus gene is in an antisense orientation relative to the regulatory region.

Another aspect of the invention is directed to a transgenic ‘Elstar’ apple cultivar comprising a gametophytic S-RNAase encoded by an S-locus gene wherein only the 3′ portion of the S-locus gene is in an antisense orientation relative to the regulatory region.

The invention is also directed to the harvestable parts, such as, flowers, fruits, ovules, pistils, ovaries, anthers, pollen and seeds as well as the progeny of the plants or trees described above, namely, the transgenic plants or trees of Malus sp., Malus domestica and ‘Elstar’ apple cultivar. The progeny of the transgenic plants or trees may be obtained by selfing the plant or tree with any of the transgenic plants or trees described herein or by crossing any of the transgenic plants or trees described herein with a plant or tree of a different genotype. The propagation material, such as, cuttings, explants, stems, shoots, roots, leaves, twigs, buds, and scions from any of the transgenic plants or trees described herein is another aspect of the invention.

Also in accordance with the present invention, there is provided a method for producing a self-compatible plant or tree selected from the group consisting essentially of Malus sp., Malus domestica, and ‘Elstar’ apple cultivar which normally exhibit self-incompatibility. The method comprises the steps of (a) introducing into a cell or tissue of Malus sp., or Malus domestica, or ‘Elstar’ apple cultivar a nucleotide sequence which is capable of down-regulating an S-locus gene, wherein the nucleotide sequence is at least one of a small interfering RNA (RNAi), an inverted repeat of an S-locus gene, or a viral sequence; (b) regenerating a plant therefrom; (c) growing the plant to a stage at which flowers are produced; (d) self-pollinating or allowing self-pollination of the flowers on the plant or tree; and selecting a self-compatible plant or tree based on the results of fruit set.

Another method of the invention is directed to a method for producing a self-compatible plant or tree of Malus sp., Malus domestica, or ‘Elstar’ apple cultivar which normally exhibits self-incompatibility. This method comprises: (a) introducing into a cell or tissue of Malus sp., Malus domestica, or ‘Elstar’ apple cultivar a nucleotide sequence which is capable of down-regulating an S-locus gene, wherein the nucleotide sequence is at least one of a small interfering RNA (RNAi), an inverted repeat of an S-locus gene, or a viral sequence; regenerating shoots therefrom; grafting the regenerated shoots onto a desirable rootstock; growing the plant or tree to a stage at which flowers are produced; self-pollinating or allowing self-pollination of the flowers on the plant or tree; and selecting a self-compatible plant or tree based on resultant fruit set.

Still another method of the invention is directed to a method for producing a self-compatible plant or tree of Malus sp., Malus domestica, or ‘Elstar’ apple cultivar which normally exhibits self-incompatibility. This method comprises the steps of: (a) introducing into a cell or tissue of Malus sp., Malus domestica, or ‘Elstar’ apple cultivar a nucleic acid molecule encoding a gametophytic S-RNAse from Malus sp, Malus domestica, or ‘Elstar’ apple cultivar respectively wherein the nucleic acid molecule encoding a gametophytic S-RNAase is in a sense or antisense orientation relative to a regulatory region which controls expression of the nucleic acid molecule; b) regenerating a plant therefrom; (c) growing the plant to a stage at which flowers are produced; (d) self-pollinating or allowing self-pollination of the flowers on the plant or tree; and (e) selecting a self-compatible plant or tree based on the results of fruit set. The gametophytic S-RNAase used in this method may be encoded by a S-locus gene, such as, S₃ or S₅ allele.

The 5′ part of the S-locus gene may be in an antisense orientation relative to the regulatory region. In the alternative, only the 3′ portion of the S-locus gene is in an antisense orientation relative to the regulatory region.

Still another aspect of the invention is directed to a method for producing a self-compatible plant or tree of Malus sp., Malus domestica, or ‘Elstar’ apple cultivar which normally exhibit self-incompatibility. This method compromises the steps of: (a) introducing into a cell or tissue of Malus sp., Malus domestica, or ‘Elstar’ apple cultivar a nucleic acid molecule encoding a gametophytic S-RNAse from Malus sp, Malus domestica, or ‘Elstar’ apple cultivar respectively and regenerating shoots therefrom; (b) grafting the regenerated shoots onto a desirable rootstock; (c) growing the plant or tree to a stage at which flowers are produced; (d) self-pollinating or allowing self-pollination of the flowers on the plant or tree; and selecting a self-compatible plant or tree based on resultant fruit set.

The gametophytic S-RNAase used in this method may be encoded by a S-locus gene, such as, S₃ or S₅ allele. The 5′ part of the S-locus gene may be in an antisense orientation relative to the regulatory region. In the alternative, only the 3′ portion of the S-locus gene is in an antisense orientation relative to the regulatory region.

Another aspect of the invention is directed to a transgenic plant or tree of Malus sp. or Malus domestica comprising tissue derived from cells transformed with an S-RNAase gene silencing construct wherein the transgenic plant or tree exhibits self-compatibility compared to a corresponding non-transformed plant or tree. The gene-silencing construct may comprise at least one of a small interfering RNA (RNAi), an inverted repeat of an S-locus gene, or a viral sequence. The S-RNAase maybe encoded by an S-locus gene and the S-locus gene may be an S₃ or S₅ allele. The harvestable parts of the transgenic plant or tree described in this paragraph, namely flowers, fruits, ovules, pistils, ovaries, anthers, pollen and seeds as well as progeny of the transgenic plant or tree are also deemed part of the invention. The progeny may be obtained by selfing the plant or tree or by crossing the plant or tree with any of the transgenic plants or trees described above to produce a plant or tree of a different genotype.

Another aspect of the invention is directed to the propagation material, namely cuttings, explants, stems, shoots, roots, leaves, twigs, buds, and scions from the plant or tree of Malus sp. comprising tissue derived from cells transformed with an S-RNAase gene silencing construct wherein the transgenic plant or tree exhibits self-compatibility compared to a corresponding non-transformed plant or tree.

S-locus genes are known and widely available. For example, any of the S-locus genes shown in FIG. 5 may be used in the methods and compositions of the present invention. Other S-locus genes, as they become available, may of course also be used in the present invention.

### BRIEF DESCRIPTION OF THE DRAWINGS

FIG. 1 is a schematic of the construction of the binary vector used for the genetic transformation of ‘Elstar’ described in the Examples. The S3 coding region was PCR-amplified and cloned into the SacI site of pFF19; the whole expression cassette was then transferred to binary vector pGPTV-KAN. 35S-prom, 35S-ter: Duplicated 35S promoter and terminator, respectively, of cauliflower mosaic virus (CaMV); Nos-prom, Ag7-ter: promoter of nopaline synthase gene and terminator of gene 7, respectively, both from Agrobacterium tumefaciens; NptII: Escherichia coli neomycin phosphotransferase gene conferring resistance to kanamycin; RB, LB: right and left border of the T-DNA, respectively.

FIG. 2A is a photograph of a transgenic apple tree revealing the self-fertility phenotype (line 81) where the upper part of a greenhouse-grown mature flowering tree is visible.

FIG. 2B is a close-up photograph of a flower from the tree of FIG. 2A, revealing the anthers (with pollen grains) and the five styles composing the pistil.

FIG. 2C is a photograph of mature fruit developed as a result of self-pollination (two upper fruits) or cross-pollination (lower left fruit) of the tree of FIG. 2A.

FIGS. 3A-3C show pollen tube growth through the style of ‘Elstar’ flowers following self-pollination in control trees and are fluorescence microscopy pictures of squash preparations of styles, stained for pollen tubes (particularly callose seen along the tube wall and in the plugs. FIG. 3A shows the top of the style just below the stigma. FIG. 3B shows halfway down the style. FIG. 3C shows the basal part of the style above the ovary (note the weakly fluorescing trichomes at the pistillar base in FIG. 3C.

FIGS. 3D-3F are photomicrographs showing pollen tube growth through the style of ‘Elstar’ flowers following self-pollination in transgenic line 81. FIG. 3D shows the top of the style just below the stigma. FIG. 3E shows halfway down the style. FIG. 3F shows the basal part of the style above the ovary.

FIG. 4 shows a western blot analysis using an antibody specific for apple S-RNAase against S-RNAse from control ‘Elstar’ trees and transgenic lines 81 and 102. Note the complete absence of the 30 kDa S-RNAse signal in the transgenic lines.

FIG. 5 shows the nucleotide sequences for various S-locus genes including the S3 and S5 alleles.

### DETAILED DESCRIPTION OF THE INVENTION

As hereinbefore described, fruit production in many fruit tree crops is dependent on cross-pollination between cultivars. This is due to the existence of a self-incompatibility (SI) mechanism. There is a strong interest in developing self-fertile fruit and nut tree crops because self-pollination would ensure more consistently high production yields compared to cross-pollination.

Presently, true self-fertile apple cultivars (Malus domestica) are commercially non-existent. The need exists therefore, for self-compatible apple plants and trees. The present invention fulfills this need by providing both methods for producing such plants and trees and the resultant compositions. The methods employ S-RNase gene silencing techniques. Such techniques include but are not limited to the use of cosuppression, antisense, small interfering RNAs (RNAi), the introduction of inverted repeats leading to dsRNA formation as well as the use of viral sequences.

Prior to this invention, it was unpredictable whether or not increasing crop loads by promoting self-compatibility in apple would be desirable since high crop loads often create lower quality fruit. In accordance with the present invention, it has been surprisingly found that fruits produced after self-pollinating the subject self-compatible plants are visually indistinguishable from fruits produced from self-incompatible apple trees of the prior art (same cultivar) after crossing.

In has also been surprisingly found that fruit set levels of the subject self-compatible plants of the present invention are comparable to fruit set levels of the self-incompatible, crossed plants of the prior art Even more surprising is the finding that fruit set levels of the self-compatible plants of the present invention remained the same even in the absence of wind or hand pollination. These results therefore demonstrate that the subject self-compatible plants do not need a pollinating vector such as bees in order to achieve successful fruit set in a commercial setting.

Thus, in accordance with the present invention, there is provided a plant or tree of Malus sp. comprising tissue derived from cells into which has been introduced a nucleotide sequence which down regulates an s-gene locus.

In another embodiment of the invention, there is provided a method for producing a self-compatible plant or tree of Malus sp., which normally exhibits self-incompatibility, said method comprising: introducing into a cell or tissue of Malus sp., a nucleotide sequence which is capable of down-regulating an S-locus gene, wherein the nucleotide sequence is at least one of a small interfering RNA (RNAi), an inverted repeat of an S-locus gene, or a viral sequence; regenerating a plant therefrom; growing the plant to a stage at which flowers are produced; self-pollinating or allowing self-pollination of the flowers on the plant or tree; and selecting a self-compatible plant or tree based on the results of fruit set.

Alternatively, the method for producing a self-compatible plant or tree of Malus sp., which normally exhibits self-incompatibility, may be performed as outlined above, except that rather than rather than regenerating a plant, shoots are regenerated and the regenerated shoots grafted onto a desirable rootstock; the plant or tree is grown to a stage at which flowers are produced; the flowers on the plant or tree are self-pollinated or allowed to self-pollinate; and a self-compatible plant or tree is selected based on resultant fruit set.

In still another embodiment of the invention, there is provided a transgenic plant or tree of Malus sp. comprising tissue derived from cells transformed with a nucleic acid molecule encoding a gametophytic S-RNAse from Malus sp, wherein the nucleic acid molecule encoding a gametophytic S-RNAase is in a sense or antisense orientation relative to a regulatory region operably linked thereto. The transgenic plant or tree exhibits self-compatibility compared to a corresponding non-transformed plant or tree.

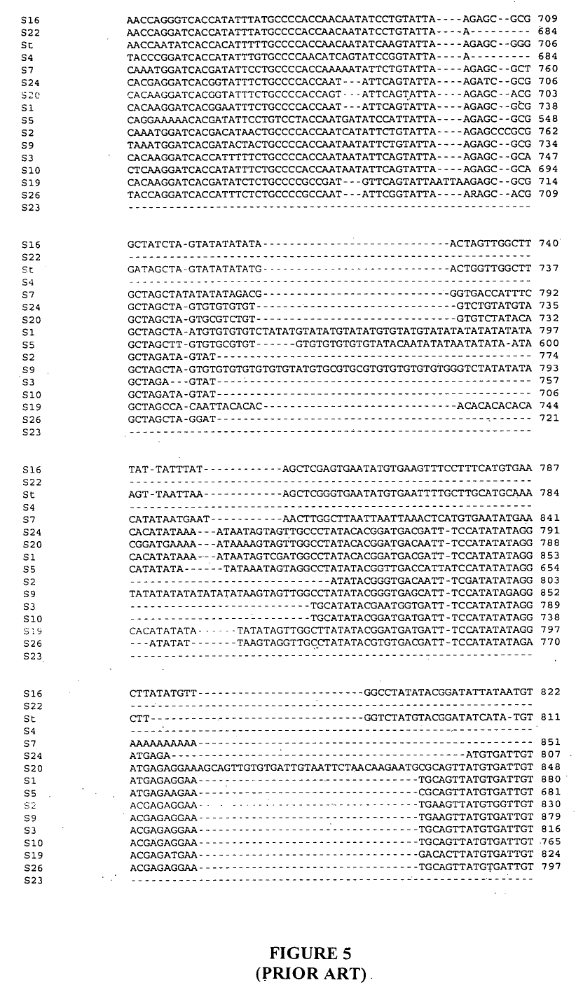

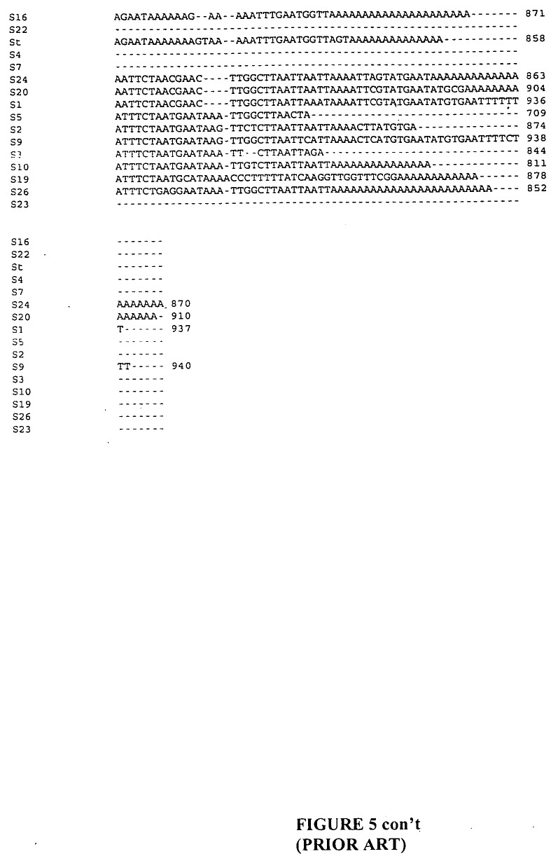

A subject plant or tree may be produced by introducing into an apple cell or tissue, a small interfering RNA (RNAi), inverted repeat, or a genetic construct comprising the gametophytic S-RNAase in a sense or antisense orientation relative to a regulatory region operably linked thereto. The regulatory region controls expression of the nucleic acid molecule and for use in the present invention, should also function in a plant cell. The gametophytic S-RNAse is known to be encoded by an S-locus gene such as the S3 or S₅ alleles. These alleles have been cloned and sequenced. See Broothaerts 2003; Broothaerts et al. 1995; Janssens et al. 1995. S-locus genes are known and widely available. The nucleotide sequences for various S-locus genes including the S3 and S5 alleles are set forth in FIG. 5.

Methods of down regulation and gene silencing via the introduction of inverted repeats leading to dsRNA formation are well known in the art, and may be found e.g., in U.S. Patent Application Publications 20030175783 and 20030049835, which publications are incorporated by reference herein as if fully set forth. The use of small interfering RNAs (RNAi) for purposes of gene silencing are also known to those of skill in the art, see e.g., Waterhouse et al. '98 “Virus Resistance and Gene Silencing in Plants can be Induced by Simultaneous Expression of Sense and Antisense RNA” Proc. Natl. Acad. Sci. 95:13959-64; Sharp, P. '99 Genes and Development 13:139-141, both of which are also incorporated by reference herein as if fully set forth. Viral sequences may also be used for downregulation and gene silencing as described in e.g., U.S. Patent Application Publication 2004/0078844, the disclosure of which is also incorporated herein by reference as if fully set forth.

There are many well-known regulatory regions which function in plant cells and plant tissue. Examples include the 35S promoter and NOS promoter, which are constitutive promoters. Other promoters such as plant cell and plant tissue- preferred promoters may also be used. Examples include: flower-specific, pistil- specific, and style-specific promoters. These promoters are widely known in the art and publicly available.

When an antisense construct is employed, preferably, only the 5′ part of the S-locus gene or alternatively, only the 3′ portion of the S-locus gene is in an antisense orientation relative to the regulatory region.

Methods of constructing the aforementioned nucleic acid molecules are well known in the art and examples of these methods fully described infra. Well-known methods of plant cell transformation and regeneration, in particular for use in Malus sp. are also available and described infra.

Harvestable parts of a subject self-compatible plant or tree are also provided. Examples include but are not limited to flowers, fruits, ovules, pistils, ovaries, anthers, pollen, and seeds.

Progeny of a subject self-compatible plant or tree is also provided by the present invention. Such progeny may be obtained by selfing a subject tree or plant or by crossing a subject plant or tree with a plant or tree of a different genotype.

The present invention further provides propagation material from a subject self-compatible plant. Examples of such materials include but are not limited to cuttings, explants, stems, shoots, roots, leaves, twigs, buds, and scions.

In another aspect of the invention, there is provided a method for producing a self-compatible plant or tree of Malus sp., which normally exhibits self-incompatibility. The method comprises the steps of: introducing into a cell or tissue of Malus sp., a nucleic acid molecule encoding a gametophytic S-RNAse from Malus sp, wherein the nucleic acid molecule encoding a gametophytic S-RNAase is in a sense or antisense orientation relative to a regulatory region which controls expression of the nucleic acid molecule; regenerating a plant therefrom; growing the plant to a stage at which flowers are produced; self-pollinating or allowing self-pollination of the flowers on the plant or tree; and selecting a self-compatible plant or tree based on resultant frit set.

In an alternative embodiment of the method, rather than regenerating a whole plant, shoots are regenerated and then grafted onto a desirable rootstock. The plant or tree is then allowed to grow to a stage at which flowers are produced. The flowers are self-pollinated or allowed to be self-pollinated and a self-compatible plant or tree selected based on resultant fruit set.

The following examples further illustrate the invention and are not meant to limit the scope thereof.

### EXAMPLE 1

### Materials and Methods

Construction of the Binary Vector

The S-gene silencing construct was derived from the S3 cDNA obtained from a style cDNA library (Broothaerts et al. 1995). PCR was used to amplify the coding sequence from the cloned S3 cDNA from start to stop codon, using primers OWB149 (50-TCTCTAGAGCTCTTGAACAAACATTATTC-30) and OWB 150 (50-ACTCTAGATGAGCTCTTAATACTG-30). After verifying the amplified product by cloning and sequence analysis, the fragment was ligated in a sense orientation into the SacI site of pFF19, between the enhanced 35S promoter and terminator sequences of cauliflower mosaic virus (CaMV) (Timmermans et al. 1991). The resulting chimaeric expression cassette was subsequently ligated into the HindIII/EcoRI restriction sites of binary vector pGPTVKAN (Becker et al. 1992), containing the nptII expression cassette near the left T-DNA border, in the opposite direction to the p35S::S3 gene cassette. The recombinant binary vector was electroporated into Agrobacterium tumefaciens strain EHA105 all of which are commercially available.

Apple Transformation

Apple leaves from in vitro shoots of cv. Elstar were co-cultivated with the A. tumefaciens strain harboring the binary vector and transgenic calli and shoots were selected on 100 mg/l kanamycin. Upon their appearance 8-16 weeks following transformation, the new shoots were elongated and propagated in vitro, followed by their transfer to ex vitro conditions by grafting the shoots directly onto the stem of an untransformed M.9 rootstock, grown in the greenhouse. Further propagation of the trees occurred by chip-budding. The potted plants were grown to maturity in a greenhouse without supplemental light or heat, except for the first growing season. Transgene insertion was verified by multiplex PCR using primers for the endogenous (intron-containing) S3-allele and both nptII and the p35S::S3 gene fusion (Broothaerts et al. 2001). In the latter case, primers were designed within the S3 cDNA sequence and in the 35S promoter region.

Pollination Assays

Greenhouse-grown trees were covered with nylon cages before flower anthesis and flowers were hand-thinned to three flowers per flower cluster. Immediately before pollination, the petals and anthers were removed from the flowers. The pollen used for pollination was derived from either untransformed ‘Elstar’ trees (self-pollination) or from Delbard Jubil, a cross-compatible variety (cross-pollination). Seven days after pollination, some flowers were removed, fixed in FAA and analyzed for pollen tube growth by fluorescence microscopy following softening in Na₂SO₃ and staining with aniline blue. Fruit set was determined in July (after June drop) and ripe fruit was picked in September, and dissected to determine seed number. Fruit set data were calculated using a minimum of five flowering trees per genotype in 2000 and one or two trees during the other years. Mean values for the 3 years are weighed, taking into account the number of flowers used. Control trees were potted untransformed ‘Elstar’ trees that were either derived from the same tissue culture used for transformation or were obtained as 2-year-old trees from a local supplier.

Western Blot Analysis

Protein extracts were prepared from five styles using the following extraction buffer: 10 mM thiourea, 100 mM Tris-HCl pH 8.5, 0.5% Tween-20, 2% polyvinylpolypyrrolidone, 14 mM b-mercaptoethanol. Proteins were separated by SDS-PAGE, followed by electroblotting onto a nylon membrane (Hybond N; Amersham, Piscataway, N.J.). Immunodetection was performed by conventional techniques, using a chicken antibody raised against the apple SRNase conserved peptide sequence CKDPPDKLFT (from H. Kokko, University of Kuopio, Finland). The secondary antibody was an anti-chicken IgG, coupled to alkaline phosphatase (Sigma, St. Louis, Mo.), and detection used the colorimetric substrate BM purple (Boehringer-Mannheim, Germany).

### EXAMPLE 2

### Production of Transgenic Apple Plants Bearing a Sense S3-Allele Construct

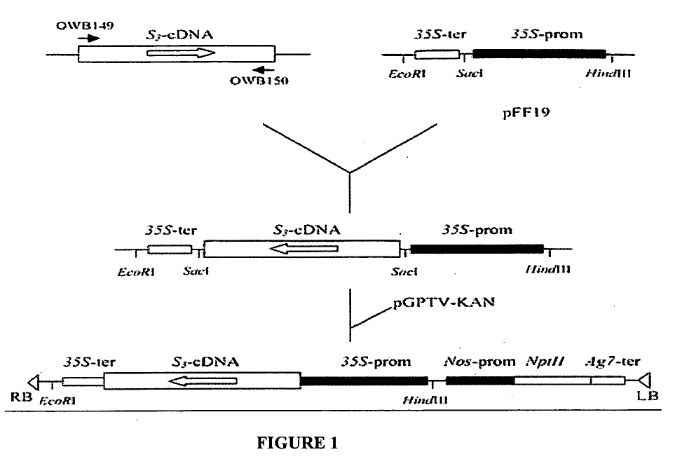

The apple variety ‘Elstar’ displays a strong SI response, as reflected by the low fruit and seed set following selfing. ‘Elstar’ bearing the alleles S₃ and S₅, and cDNAs for both S-alleles have been cloned (Broothaerts et al. 1995; Janssens et al. 1995). S₃ is by far the most common S-allele within the domesticated apple species and the presence of its gene product in the pistil has been confirmed by in situ immuno-localization studies, applying an S₃-peptide-specific antibody (Certal et al. 1999). Based on the S₃ cDNA, a co-suppression construct was prepared for the transformation of ‘Elstar’. This constrict contained the full-length sense S₃ cDNA sequence driven by the double CaMV 35S promoter and is shown in FIG. 1. From 1,500 in vitro leaf explants co-cultivated with A. tumefaciens, 13 independent transgenic plants were produced. These lines were shown to contain an intact S-transgene and nptII gene by PCR. Based on preliminary pollination experiments (see below), transformation events 81 and 102 were chosen for further analysis. Copy number analysis by Southern blotting revealed the integration of T-DNA copies at six and two genomic locations. The in vitro grown shoots of these two transgenic lines weakly transcribe the transgene S₃ mRNA, as well as producing hybridization signals of a much larger size (Janssens 1997). It is noted that the targeted endogenous S-gene is expressed only in the pistil of the flower and not in any other tissues (Broothaerts et al. 1995).

### EXAMPLE 3

### Controlled Pollination Tests

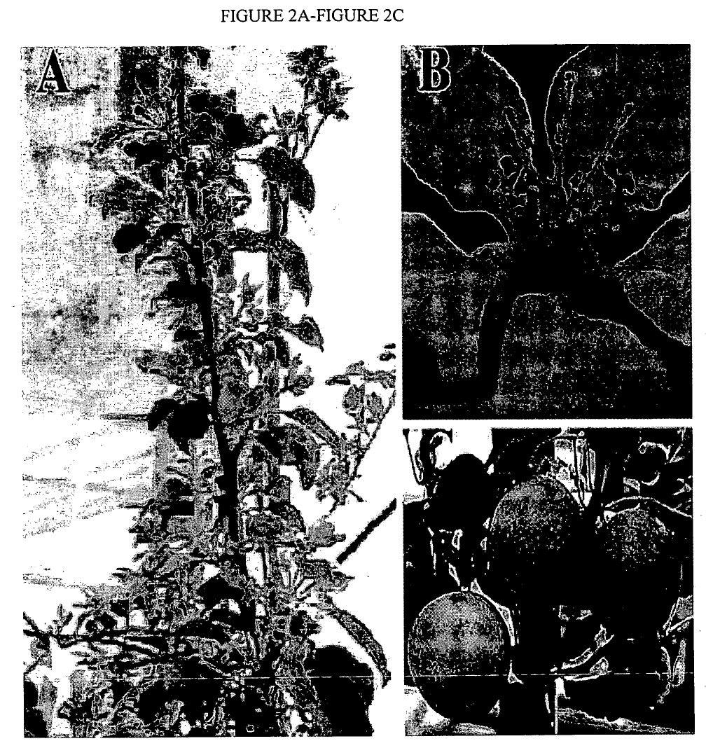

Plants from transgenic lines 81 and 102 were grown to maturity in the greenhouse and a are shown in FIG. 2A. These plants were shown to bear normal fertile flowers and are shown in FIG. 2B. As shown in FIG. 2B the normal fertile flowers have anthers (with pollen grains) and five styles composing the pistil. These plants were analyzed for SI by hand-pollination. Some of the flowers were self-pollinated (using pollen from control ‘Elstar’ trees), and some were cross-pollinated with a compatible pollen donor variety (Delbard Jubil, bearing the S-alleles S2 and S22; Broothaerts 2003). This experiment was repeated during the following 2 years and the results are summarized in Table 1 below.

TABLE 1

| Year | Pollination | Line 81 | | Line 102 | | Control | |
|---|---|---|---|---|---|---|---|
| 1999 | Cross | 8 | (97) | 22 | (58) | 24 | (762) |
| | Self | 5 | (161) | 19 | (63) | 4 | (1,068) |
| 2000 | Cross | 40 | (287) | 29 | (266) | 51 | (620) |
| | Self | 47 | (346) | 26 | (331) | 6 | (688) |
| 2001 | Cross | 24 | (80) | 16 | (112) | 21 | (1,035) |
| | Self | 24 | (86) | 5 | (123) | 2 | (1,002) |
| 1999-2001 | Cross | 31 | (464) | 25 | (436) | 30 | (2,417) |
| | Self | 32 | (593) | 20 | (547) | 4 | (2,758) |

As shown in Table 1, in the control ‘Elstar’ trees, the mean fruit set following self-pollination during 1999-2001 was 4%, while fruit setting after cross-pollination increased to 30%. In line 81, the mean fruit set following both self and cross-pollination was identical (31-32%), indicating that both pollen sources were equally efficient at fertilization. Similar results were obtained for line 102. Using transgenic pollen from either transgenic line for pollination of the control or the transgenic flowers gave comparable results to those obtained when using pollen from untransformed trees. This indicates that the transformation has not affected the SI behavior of the pollen, i.e. they were arrested in an incompatible style and grew without restriction in a compatible (transgenic) style. Some variation in the fruit setting was observed from year to year, which was likely caused by differences in tree maturity or vigor and in the environmental conditions in the greenhouse, e.g. in 2000 temperatures were generally higher during and after flowering, which promoted fruit setting.

The fruits produced after selfing the transgenic genotypes were visually indistinguishable from the ones produced after crossing as shown in FIG. 2C. The two upper fruit shown in FIG. 2C were the result of self-pollination and the lower left fruit was the result of cross-pollination. In addition, the number of seeds of the fruit that were the result of self-pollination and the fruit that were the result of cross-pollination was similar in both cases. The mean average of seeds in both cases was seven seeds per fruit.

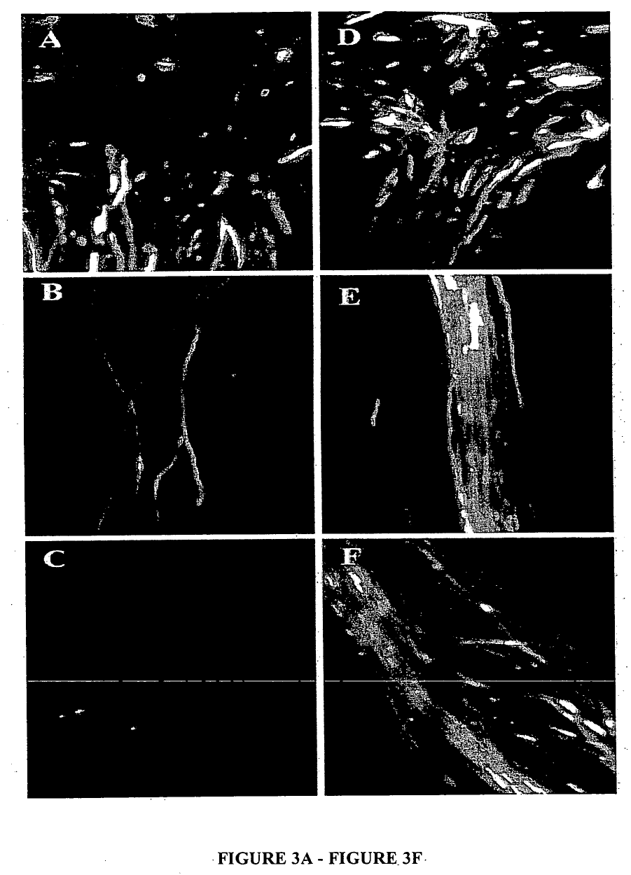

In contrast, fruit resulting from selfing contained roughly only half the number of seeds per fruit compared to fruit resulting from cross-pollination on the control trees. This is further evidence that the SI mechanism was not operational in the transgenic trees, while it functioned in the control trees, albeit not completely effectively. Further evidence for the inactivation of the SI mechanism also came from fluorescence microscopic analysis of pollen tube growth through the pistil as shown in FIG. 3.

In control trees, self-pollen was never observed near the base of the pistil, while cross-pollen was abundantly present in that region at the time of analysis (7 days after hand pollination). In the transgenic plants, both self- and cross-pollen grew efficiently through the styles, and pollen tubes were found at the stylar bases in both cases. This indicates that pollen tube growth following self-pollination in the transgenic trees was apparently not restricted by the SI mechanism.

### EXAMPLE 4

### S-RNase Expression in the Pistil

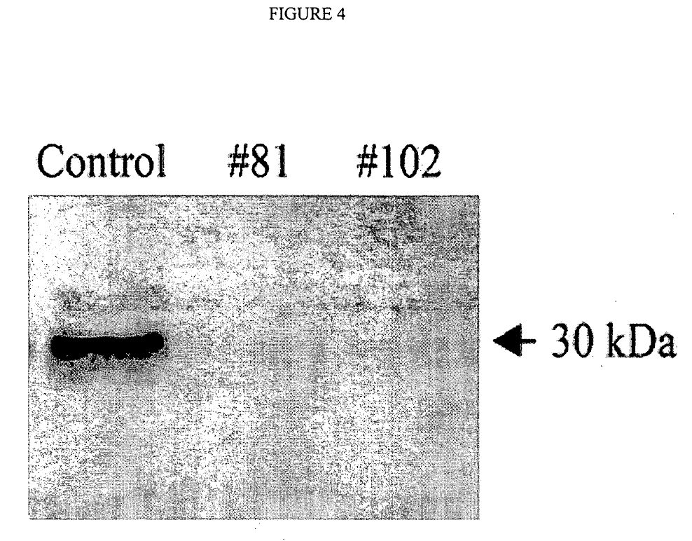

To investigate if the absence of a functional SI mechanism in the transgenic trees was the result of inhibition of SRNase expression, protein extracts of styles were separated by electrophoresis and blotted. An anti-S-Rnase antibody raised against a conserved peptide was used to detect the apple S-RNases. The control styles clearly showed the presence of the pistil S-RNases, whereas the transgenic pistils were completely devoid of them as shown in FIG. 4.

As the separation method employed did not discriminate between the two ‘Elstar’ S-RNases (S3 and S5), this result reveals that the transgene employed not only inhibited the expression of the targeted S3-allele, but also that of the S5-allele. From native-PAGE and segregation experiments, we know that both S-RNases are expressed in wild-type ‘Elstar’ pistils. S-allele-specific PCR analysis of segregating seedlings following self-pollination of the transgenic lines confirmed that fertilization resulted from both S3 and S5 pollen grains.

As shown by Western blot staining, the S-RNase signal was completely absent from lanes loaded with protein extract from the transgenic pistils. This indicates that the transgene has resulted in a complete, or at least significant, down regulation of S3-RNase expression. Moreover, because of their apparent similarity, the gel analysis system employed did not resolve the S3- and S5-RNases. The absence of any S-RNase signal on the blot, therefore, indicated that both S-RNase alleles were rendered nonfunctional, despite the use of a cDNA transgene derived from the S3-RNase gene only. The S3 and S5-allele sequence are 77% identical in their coding region. Presumably, this homology has caused the silencing of both endogenous alleles in the transgenic apple trees analyzed. In three out of six silenced Petunia inflata plants, both S-alleles had been rendered non-functional, while in the remaining three plants only the S-allele corresponding to the targeted allele was affected (Lee et al. 1994). In these experiments, an antisense S-allele sequence that was driven by its own promoter was used. The homology between the S-alleles affected in the transgenic Petunia plants approximated 80% for the full coding region, but the antisense transgene was only composed of approximately 70% of this region.

### EXAMPLE 5

### Development of Transgenic Self-Compatible Lines with Alternative Constructs

Several lines were analyzed in addition to the lines described above containing the full length S3 gene in the sense orientation used to achieve co-suppression. These additional lines include: A) a construct with the full length S3-gene in antisense orientation yielding 10 transgenic lines: 1-3-6-9-12-13-16-19-22-23; B)a construct with the 5′ part of S3 in antisense orientation yielding 9 transgenic lines: 26-28-31-33-36-39-42-44-45; C)a construct with the 3′ part of S3 in antisense orientation yielding 15 transgenic lines: 47-50-53-54-55-58-59-61-64-66-68-71-72-74-77; and D) the construct described above, namely 78-81-84-87-88-90-91-94-97-99-102-103-106-107.

Additional Western blot analysis for additional lines compared to the construct described above, namely 78-81-84-87-88-90-91-94-97-99-102-103-106-107 (Described in Broothaerts et al., 2004).

The experiment was done for 46 lines (1-3-6-9-12-13-16-19-22-23-26-28-31-33-36-39-42-44-45-47-50-53-54-55-58-59-61-64-66-68-71-72-74-77-78-84-87-88-90-91-94-97-99-103-106-107). Results obtained here were used as a guide to discriminate between “self-fertile” lines, “partially self-fertile” lines and “wild type” lines. These lines are labeled as follows: A) Self-fertile: 39-44-45-78-81-102 (bold: published by Broothaerts et al. 2004); B)Partially self-fertile: 16-26-47-50-53-55-58-59-66-72; and C)Wild type (no silencing): 1-3-6-9-12-13-19-22-23-28-31-33-36-42-54-61-64-68-71-74-77.

Based on these additional results one can conclude that the strategy using a T-DNA with the full length S3 gene in antisense orientation did not yield any lines with complete silencing and was the least successful strategy. The approach using the 5′ part of the S3 gene in antisense was as successful as the approach described above (and in Broothaerts et al., 2004), namely, using the full length S3 gene in the sense orientation in order to achieve co-suppression. The results obtained by the strategy using 3′ part of S3 in anti-sense was intermediate successful. These results are derived from Western blots using a chicken antibody raised against the apple S-RNase conserved peptide sequence CKDPPDKLFT. The secondary antibody was an anti-chicken IgG, coupled to alkaline phosphatase.

Analysis of Silencing of the S3-Gene on the Transcriptional Level

The technique used was semi-quantitative RT-PCR analysis. This was done for 13 independent transgenic lines (39-45-47-50-53-55-58-59-66-72-78-81-102). The results from these experiments confirmed the Western blotting results.

Analysis of the Integration Pattern of the T-DNA

This was done by Southern blotting experiments using different restriction digests and probes. Analysis was performed for all transgenic lines obtained by the different transformation strategies (cfr. list above, paragraph 1a).

In most lines more than one copy of the T-DNA was inserted. Remarkably, all the lines where S3-silencing occurs (except lines 45-50-72) carry an inverted repeat around the Right Border (this is the side of the T-DNA in which the S3-gene is situated). We have Northern blot results with leaf material which indicate that read-through transcription occurs, explaining the silencing (inverted repeat structure).

Analysis of Heredity of the T-DNA Construct in an F1-Population

Lines 39-53-66-102 were selfed and cross-pollinated with the compatible cultivar Delbard Jubilé (S2S24). All seedlings resulting from selfing and cross-pollination were examined for the presence of T-DNA. This was done by two strategies: PCR detection of the marker gene (nptII) and the transferred S3 sequence (for all seedlings) on one hand and Southern analysis (for 25% of the seedlings) on the other hand. It could be concluded that for the lines 53, 66 and 102 the T-DNA trait inherits in a Mendelian way (as one locus). This was expected in combination with the results of the Southern blotting for the estimation of the integration pattern. For line 39, inheritance is not Mendelian, which could also be expected based on the Southern results (line 39 carries 7 copies of the T-DNA). The results arising from the Southern analysis show that in lines 39, 53 and 102 the integration pattern remains stable. In the progeny of line 39 often a segregation of the insertion pattern is observed.

Analysis of the S-Genotype of an F1-Population

This was done on the seedlings described in the former paragraph. By PCR analysis the possible presence of the S3 and S5 alleles (S-genotype “Elstar”: S3S5) was examined. For the seedlings resulting after cross-pollination in addition the presence of S2 and S24 allele was investigated. The S-allele genotypes of the progeny from cross pollination served to estimate the equality of fitness for all pollen grains in order not to misinterpret the inheritance of the S3 and S5 alleles after selfing. For all lines except line 53 (see also further, paragraph 2b.) the pollen grains were equally fit, meaning that differences in inheritance arise from the situation in the pollen tube and not from bad pollen quality.

The results show clearly that the S3 gene was silenced compared to S5, since the S3-allele is significantly more inherited than the S5 allele in all lines tested.

Microscopical Analysis of Pollen Tube Growth Within the Style in Transgenic Versus Control Plants

This was analyzed in fixated styles by means of aniline blue staining which colors the callose component of the cell wall of growing pollen tubes. This was done for the transgenic lines 39, 53, 66 and 102. After 5 days there is a significant difference of pollen tube growth after selfing in the transgenic when compared to the wild type “Elstar” control: in all lines the pollen tubes have reached the base of the style and have entered the ovules whereas in the control pollen tube growth is inhibited in the upper part of the style, hereby impeding successful pollination.

### EXAMPLE 6

### Controlled Pollination Tests Under Greenhouse Conditions

In the Greenhouse Trials of 2002 and 2003 the Effect of “No Pollination” on Fruit Set was Tested

The ‘selfing’ experiments described above for the full length S3 gene in the sense orientation used to achieve co-suppression, (and in Broothaerts et al. (2004), concerned hand-pollination of transgenic flowers with pollen from non-transgenic flowers of the same genotype. In a commercial orchard, self-pollination would have to occur without human interference to be of agronomical relevance. Therefore, the effect of “no pollination” was studied. This means that no pollen was brought on the pistils of the transgenic trees. Wind pollination was nihil since there was no wind flow in the greenhouse. We observed fruit set levels comparable to cross pollination, meaning that self fertile plants do not need a vector (such as bees) in order to achieve successful fruit set. These observations establish that S-RNase gene silencing can be used to obtain self-fertility in an agronomically relevant way.

All references referred to in this application are herein incorporated by reference in their entirety.

While the invention has been described with reference to certain embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out the process of the invention but that the invention will include all embodiments falling within the scope of the appended claims.

### REFERENCES

Becker D, Kemper E, Schell J, Materson R (1992) New plant binary vectors with selectable markers located proximal to the left T-DNA border. Plant Mol Biol 20:1195-1197.

Broothaerts W (2003) New findings in apple S-genotype analysis resolve previous confusion and request the re-numbering of some S-alleles. Theor Appl Genet 106:703-714.

Broothaerts W., et al. (2004) Self-fertile apple resulting from S-Rnase gene silencing. Plant Cell Reports 22:497-501.

Broothaerts W, Janssens G A, Proost P, Broekaert W F (1995) cDNA cloning and molecular analysis of two self-incompatibility alleles from apple. Plant Mol Biol 27:499-511.

Broothaerts W, Wiersma P A, Lane W D (2001) Multiplex PCR combining transgene and S-allele control primers to simultaneously confirm cultivar identity and transformation in apple. Plant Cell Rep 20:349-353.

Certal A C, Sanchez A M, Kokko H, Broothaerts W, Oliveira M M, Feij_J A (1999) S-RNase in apple are expressed in the pistil along the pollen tube growth path. Sex Plant Reprod 12:94-98.

Godini A, Palasciano M, Cozzi G, Petruzzi G (1998) Role of self pollination and horticultural importance of self-compatibility in cherry. Acta Hortic 468:567-574

Goldway M, Shai O, Yehuda H, Matityahu A, Stern R A (1999). ‘Jonathan’ apple is a lower-potency pollenizer of ‘Topred’ than ‘Golden Delicious’ due to partial S-allele incompatibility. J Hortic Sci Biotechnol 74:381-385.

Golz J F, Oh H-Y, Su V, Kusaba M, Newbigin E (2001) Genetic analysis of Nicotiana pollen-part mutants is consistent with the presence of an S-ribonuclease inhibitor at the S-locus. Proc Natl Acad Sci USA 98:15372-15376.

Igic B, Kohn J R (2001) Evolutionary relationships among self incompatibility RNases. Proc Natl Acad Sci USA 98:13167-13171.

Janssens G (1997) Molecular analysis of the self-incompatibility(S-) gene in apple (Malus x domestica Borkh.). S-genotyping of apple cultivars and (anti)sense suppression of the S-gene. PhD dissertation no. 350, Fac Agric Appl Biol Sci, KatholiekeUniversiteit Leuven, Belgium.

Janssens G A, Goderis I J, Broekaert W F, Broothaerts W (1995) A molecular method for S-allele identification in apple based on allele-specific PCR. Theor Appl Genet 91:691-698.

Lee H-S, Huang S, Kao T-h (1994) S-proteins control rejection of incompatible pollen in Petunia inflata. Nature 367:560-563.

Murfett J, Atherton T L, Mou B, Gasser C S, McClure B A (1994) SRNase expressed in transgenic Nicotiana causes S-allele specific pollen rejection. Nature 367:563-566.

Muskens M W M, Vissers A P A, Mol J N M (2000) Role of inverted DNA repeats in transcriptional and post-transcriptional gene silencing. Plant Mol Biol 43:243-260.

Nettancourt D de (2001) Incompatibility and incongruity in wild and cultivated plants. Springer, Berlin Heidelberg New York Sassa H, Hirano H, Nishio T, Koba T (1997) Style-specific self compatible mutation caused by deletion of the S-RNase gene in Japanese pear (Pyrus serotina). Plant J 12:223-227.

Smith N A, Singh S P, Wang M-B, Stoutjesdijk P A, Green A G, Waterhouse P M (2000) Total silencing by intron-spliced hairpin RNAs. Nature 407:319-320.

Timmermans M C P, Maliga P, Vieira J, Messing J (1991) The pFF plasmids: cassettes utilizing CaMV sequences for expression of foreign genes in plants. J Biotechnol 14:333-344 501.

1. A transgenic plant or tree of Malus sp. comprising tissue derived from cells transformed with a nucleic acid molecule encoding a gametophytic S-RNAse from Malus sp, wherein the nucleic acid molecule encoding a gametophytic S-RNAase is in a sense or antisense orientation relative to a regulatory region operably linked thereto, said regulatory region controlling expression of the nucleic acid molecule, and wherein the transgenic plant or tree exhibits self-compatibility compared to a corresponding non-transformed plant or tree.

2. The plant or tree of claim 1 wherein the gametophytic S-RNAase is encoded by an S-locus gene.

3. The plant or tree of claim 2 wherein the S-locus gene is an S₃ or S₅ allele.

4. The plant or tree of claim 2 wherein only the 5′ part of the S-locus gene is in an antisense orientation relative to the regulatory region.

5. The plant or tree of claim 2 wherein only the 3′ portion of the S-locus gene is in an antisense orientation relative to the regulatory region.

6. The plant or tree of claim 2 wherein the S-locus gene has the nucleotide sequence as set forth in FIG. 5.

7. The plant or tree of claim 1 which is Malus domestica.

8. The plant or tree of claim 5 which is the ‘Elstar’ apple cultivar.

9. Harvestable parts of the plant or tree of any of claims 1-8.

10. The harvestable parts of the plant or tree of claim 9 selected from the group consisting of flowers, fruits, ovules, pistils, ovaries, anthers, pollen and seeds.

11. Progeny of the plant or tree of any of claims 1-8.

12. Progeny obtained by selfing the plant or tree of any of claims 1-5.

13. Progeny obtained by crossing the plant or tree of any of claims 1-5 with a plant or tree of a different genotype.

14. Propagation material from the plant or tree of any of claims 1-8.

15. The propagation material of claim 14 selected from the group consisting of cuttings, explants, stems, shoots, roots, leaves, twigs, buds, and scions.

16. A method for producing a self-compatible plant or tree of Malus sp., which normally exhibits self-incompatibility, said method comprising:
(a) introducing into a cell or tissue of Malus sp., a nucleotide sequence which is capable of down-regulating an S-locus gene, wherein the nucleotide sequence is at least one of a small interfering RNA (RNAi), an inverted repeat of an S-locus gene, or a viral sequence;
(b) regenerating a plant therefrom;
(c) growing the plant to a stage at which flowers are produced;
(d) self-pollinating or allowing self-pollination of the flowers on the plant or tree; and
(e) selecting a self-compatible plant or tree based on the results of fruit set.

17. A method for producing a self-compatible plant or tree of Malus sp., which normally exhibits self-incompatibility, said method comprising:
(a) introducing into a cell or tissue of Malus sp., a nucleotide sequence which is capable of down-regulating an S-locus gene, wherein the nucleotide sequence is at least one of a small interfering RNA (RNAi), an inverted repeat of an S-locus gene, or a viral sequence;
(b) regenerating shoots therefrom;
(c) grafting the regenerated shoots onto a desirable rootstock;
growing the plant or tree to a stage at which flowers are produced;
self-pollinating or allowing self-pollination of the flowers on the plant or tree; and
selecting a self-compatible plant or tree based on resultant fruit set.

18. A method for producing a self-compatible plant or tree of Malus sp., which normally exhibits self-incompatibility, said method comprising:
(a) introducing into a cell or tissue of Malus sp., a nucleic acid molecule encoding a gametophytic S-RNAse from Malus sp, wherein the nucleic acid molecule encoding a gametophytic S-RNAase is in a sense or antisense orientation relative to a regulatory region which controls expression of the nucleic acid molecule;
(b) regenerating a plant therefrom;
(c) growing the plant to a stage at which flowers are produced;
(d) self-pollinating or allowing self-pollination of the flowers on the plant or tree; and
(e) selecting a self-compatible plant or tree based on the results of fruit set.

19. A method for producing a self-compatible plant or tree of Malus sp., which normally exhibits self-incompatibility, said method comprising:
(a) introducing into a cell or tissue of Malus sp., a nucleic acid molecule encoding a gametophytic S-RNAse from Malus sp, regenerating shoots therefrom;
(b) grafting the regenerated shoots onto a desirable rootstock;
(c) growing the plant or tree to a stage at which flowers are produced;
(d) self-pollinating or allowing self-pollination of the flowers on the plant or tree; and
(e) selecting a self-compatible plant or tree based on resultant fruit set.

20. The method of claim 18 or 19 wherein the gametophytic S-RNAase is encoded by an S-locus gene.

21. The method of claim 20 wherein the S-locus gene is an S₃ or S₅ allele.

22. The method of claim 21 wherein the S-locus gene has the nucleotide sequence as set forth in FIG. 5.

23. The method of any of claims 16-19 wherein the plant or tree is Malus domestica.

24. The method of claim 23 wherein the plant or tree is the ‘Elstar’ apple cultivar.

25. The method of claim 18 or 19 wherein only the 5′ part of the S-locus gene is in an antisense orientation relative to the regulatory region.

26. The method of claim 18 or 19 wherein only the 3′ portion of the S-locus gene is in an antisense orientation relative to the regulatory region.

27-38. (canceled)
